(19) United States
(12) Patent Application Publication
(10) Pub. No.: US 2020/0123606 A1
(43) Pub. Date: Apr. 23, 2020

(54) RT-QPCR METHOD FOR DIRECT QUANTITATIVE DETECTION OF CIRCULATING MIRNA

(71) Applicant: SHENZHEN UNIVERSITY, Nanshan Shenzhen, Guangdong (CN)

(72) Inventors: Deming GOU, Nanshan Shenzhen, Guangdong (CN); Yanqin NIU, Nanshan Shenzhen, Guangdong (CN); Kang KANG, Nanshan Shenzhen, Guangdong (CN)

(73) Assignee: SHENZHEN UNIVERSITY, Nanshan Shenzhen, Guangdong (CN)

(21) Appl. No.: 16/622,122

(22) PCT Filed: Dec. 20, 2017
(86) PCT No.: PCT/CN2017/117558

§ 371 Date: Dec. 12, 2019

(30) Foreign Application Priority Data
Jun. 13, 2017 (CN) 201710442989.5

(51) Int. Cl. C12Q 1/6876; C12Q 1/686

(52) U.S. Cl. CPC C12Q 1/6876; C12Q 1/686

(57) ABSTRACT

A real-time fluorescence quantitative RT-qPCR method for direct detection of circulating miRNAs in serum or plasma without the need of extracting nucleic acids. Said method comprises: S1, subjecting cleavages of exosomes and miRNA-protein complexes in serum or plasma, and performing centrifugation to obtain a crude circulating miRNA extract; S2, performing miRNA tailing and reverse transcription; and S3, performing RT-qPCR quantitative detection. Said method does not required nucleic acids to be extracted, and Poly(A) tailing and reverse transcription of miRNA will be synchronously accomplished in one reaction system. The operation is simple, the time is shortened, and the preparation of cDNAs is completed within 95 minutes. Compared with the stem-loop method, said method provides a sensitivity increased by several tens or even hundreds of times, establishes a very simple, sensitive, efficient, fast and inexpensive miRNA detection technology system, and is especially suitable for clinical application and for the detection of miRNAs from biological fluid samples having low miRNA abundance.

### TECHNICAL FIELD

The present disclosure relates to the field of biomedicine technology, and in particular to an RT-qPCR method for directly and quantitatively detecting circulating miRNAs without extracting nucleic acids.

### BACKGROUND

MicroRNAs (miRNAs) are a class of non-coding small RNAs of about 22 nucleotides in length and are widely found in eukaryotes such as animals, plants, and nematodes. miRNA degrades target mRNA or prevents its translation by binding to the 3′ untranslated region (3′-UTR) of the target mRNA, and thereby regulates gene expression at the post-transcriptional level. Functionally, miRNAs are widely involved in cell differentiation, proliferation, apoptosis, individual growth and development, and organ formation. The expression of miRNA is finely regulated in organisms and has strict time and space specificity. Studies have shown that circulating miRNAs are present in blood and they are very stable therein. More importantly, abnormalities in circulating miRNAs are closely related to the occurrence and development of many diseases, and thus circulating miRNAs can be used as novel biomarkers for early diagnosis and prognosis evaluation of major diseases such as cancer.

Real-time quantitative PCR (RT-qPCR)-based detection has been considered as one of the most sensitive miRNA detection methods for a long time. Commonly used methods include a poly(A) tailing method (Shi R, Biotechnique. 2005, 39 (4): 519-525) and a stem-loop method (Chen C, Nucleic Acids Res. 2005, 33(20): 1-9). The Poly(A) tailing method is performed by adding a poly(A) tail to the 3′ end of miRNA with a poly(A) polymerase, and then reverse-transcribing the miRNA with a primer containing an Oligo (dT) sequence. Due to universality of the primer used in the reverse transcription, the Poly(A) tailing method not only has a reduced detection cost, but also has a decreased detection specificity and sensitivity. In the stem-loop method, the primer used in the reverse transcription contains a stem-loop structure at the 5′ end, and usually contains 6 specific bases complementary to the 3′ end of the miRNA at the 3′ end so that the primer can be used in a specific reverse transcription reaction. However, due to sequence-specific probes used in the stem-loop method, a high-throughput miRNA analysis with this method is relatively high cost. In addition, the binding strength generated from the matching of six bases is obviously insufficient, and thus the efficiency of cDNA synthesis is significantly reduced.

Kang K et al. developed a novel method for detecting miRNAs, S-Poly (T) method, which is disclosed in Patent Application No. CN102154505A (it is referred to as the S-Oligo(dT) method in this patent, and the primer used therein is referred to as a S-Oligo(dT) primer) and in Kang, K, PloS one. 2012. 7, e48536. In the S-Poly(T) method, the used primer is composed of, in order from the 5′ end, a universal PCR primer sequence of 14 to 20 nucleotides in length, a universal probe sequence of 14 to 20 nucleotides in length, 8 to 30 dTs, and a specific sequence complementary to 3 to 8 nucleotides at the 3′ end of the target miRNA. Compared with the poly(A) tailing method and the stem-loop method, the S-Poly(T) method has greatly improved specificity and sensitivity, wherein the sensitivity is improved by at least 10-fold. In an upgraded version of the S-Poly (T) method, S-Poly(T) Plus method (Patent Application Number: 201510558101.5), the Poly(A) tailing and reverse transcription of the miRNA is carried out in an one-step reaction. Therefore, in terms of convenience of operation and efficiency of reverse transcription, the S-Poly (T) Plus technique has been further modified and improved, and its overall sensitivity is 2-8 times higher than the S-Poly(T) method.

Current miRNA detection methods are based on purified RNA as a template. Due to incomplete precipitation and recovery of RNA during nucleic acid extraction, some RNA will inevitably be lost. In addition, the RNA extraction process is time consuming and prone to contamination and degradation.

It can be seen that the prior art needs to be improved.

### SUMMARY

In view of problems described above, it is desired to provide a RT-qPCR-based method for directly and quantitatively detecting a circulating miRNA, which is simpler and more sensitive, efficient, and inexpensive, and is referred to Direct S-Poly(T) Plus (DSPP) herein.

In order to achieve the purpose described above, the present disclosure comprises the following technical solutions:

A RT-qPCR-based method for directly and quantitatively detecting a circulating miRNA, in which the miRNA is lysed from the protein complex and directly used in RT-qPCR detection without extracting and purifying nucleic acids.

Further, the RT-qPCR-based method for directly and quantitatively detecting a circulating miRNA comprises the following steps:

S1, lysis and centrifugation: fully lysing the protein complex in a sample with a lysis reagent to release the miRNA from the protein complex in the sample; centrifuging the resultant mixture to obtain a supernatant as an extracted crude RNA, wherein about 35 μl supernatant can be obtained from 40 μl of the mixture;

S2, tailing and reverse transcription: adding a Poly(A) tail to the extracted crude RNA obtained in step S1 and performing S-Poly (T) specific reverse transcription;

S3, RT-qPCR quantitative detection: performing RT-qPCR quantitative detection by using the reverse transcription product cDNA obtained in step S2 as a template.

Further, the amount of the sample in step S1 is 20 to 50 μl.

Further, the lysis reagent in step S1 comprises components: 20 μl of 2×lysis buffer, 1 μl of protease K, and is used to process 20 μl of the sample.

Further, the 2×lysis buffer comprises the following components at their final concentrations: 100 mmol/1 Tris-HCl, 300 mmol/l NaCl, 20 mmol/1 MgCl₂; pH 8.0.

Further, the final concentration of the proteinase K is 15 U/mL.

Further, the lysis is carried out at 50° C. for 20 minutes and then at 95° C. for 5 minutes.

Further, the centrifugation in step S1 is carried out at 10,000 to 14,000 g, 4° C. for 5 to 15 minutes; preferably, at 13,000 g, 4° C. for 5 minutes.

Further, the reaction system for the tailing and reverse transcription in step S2 comprises polyA polymerase and reverse transcriptase.

Further, the extracted crude RNA as a template used in the reaction system for the tailing and reverse transcription in step S2 has a volume percentage of 5 to 75%, preferably 40%.

Further preferably, the reaction system for the tailing and reverse transcription comprises: 0.5-7.5 μL of the supernatant as a template, 1±0.2 μL of 0.5 μmol/L RT primer, 1±0.2 U of PolyA Polymerase, 100±20 U of MMLV, and 2.375-0.625 μl of reaction buffer, and supplement of RNase-free Water to 10 μL; and the tailing and reverse transcription is carried out by incubating the reaction system at 37-42° C. for 50-70 min, incubating the reaction system at 74-76° C. for 3-7 min to inactivate the enzymes, and then quickly placing the reaction system on ice and allowing the same to stand for 2 min to terminate the inactivation.

Further preferably, the reaction system for the tailing and reverse transcription comprises: 4 μl of the supernatant as a template, 1 μL of 0.5 μM RT primer, 1 U of PolyA Polymerase, 100 U of MMLV, 1.5 μL of reaction buffer, and supplement of RNase-free Water to 10 μL; and the tailing and reverse transcription is carried out by incubating the reaction system at 37° C. for 30 min, incubating the reaction system at 75° C. for 5 min to inactivate the enzymes, and then quickly placing the reaction system on ice and allowing the same to stand for 2 min to terminate the inactivation.

Further, cDNA is used as a template for the real-time PCR quantitative detection in step S3 and a hot-start DNA polymerase is used in this process to reduce non-specific amplification. The reaction system for the real-time PCR consists of: 5 μL of 4× qPCR reaction Buffer, 4 μL of 1 μmol/L Forward Primer, 0.4 μL of 10 μmol/L universal reverse primer, 0.5 μL of 10 μmol/L universal Taqman probe, 0.2 μL of 100×ROX Reference Dye, 0.0125 μL of Hotstart Alpha Taq Polymerase, 0.5 μL of cDNA, and supplement of RNase-free Water to 20 μL; the real-time PCR is carried out as follows: 40 cycles of pre-denaturation at 95° C. for 5 minutes, denaturation at 95° C. for 10 s, and annealing at 60° C. for 40 s.

Further, the hot-start enzyme is prepared by mixing the DNA polymerase and the hot-start antibody in equal volumes and allowing the mixture to stand at room temperature for 6 hours.

Further, the sample is selected from serum, plasma/serum, urine, tears, milk, saliva, sputum, or stool extraction supernatant; and preferably the sample is plasma.

The present disclosure has the following beneficial effects:

1. In the Direct S-Poly(T) Plus method of the present disclosure, miRNA can be quantitatively detected without a step of extracting nucleic acids. The flow chart thereof is shown in FIG. 1. The method has the advantages of convenient operation and shortened time period, wherein the period for preparing cDNA is reduced by at least 70%, and the simplicity thereof is superior to conventional methods.

2. In the Direct S-Poly(T) Plus method of the present disclosure, the amount range of the template required by the reverse transcription is broader. 5% to 75% of the extracted crude RNA can meet the requirements for the reverse transcription. The transcription efficiency is superior to conventional methods.

3. The technical system of the present disclosure is particularly suitable for detecting miRNAs from biological fluid samples having low miRNA abundance. The sensitivity of the method of the present disclosure is significantly higher than that of conventional methods. For example, in terms of sensitivity, 175 miRNAs can be detected by using only 20 μl of a body fluid sample.

4. The Direct S-Poly(T) Plus method of the present disclosure can efficiently detect miRNAs from biological fluid samples including serum, plasma/serum, urine, milk, saliva, sputum, stool extraction supernatant, and cell culture. The detection efficiency thereof is an order of magnitude higher than conventional methods, thereby improving the sensitivity and accuracy of miRNA quantitative detection in a body fluid sample.

5. Due to the simplicity, sensitivity, and specificity, the method of the present disclosure shows promising application prospects in early screening and prognosis evaluation of diseases, and can be widely used in early non-invasive screening of tumors, cardiovascular diseases or other major diseases.

### BRIEF DESCRIPTION OF THE DRAWINGS

FIG. 1 shows the flow chat of the miRNA direct RT-qPCR fluorescence quantitative detection (Direct S-Poly(T) Plus). Wherein, in the system for the tailing and reverse transcription, as an optimal embodiment, 4 μl of an extracted crude RNA is used as a template.

FIG. 2 shows comparison of effects of different lysis schemes used in the Direct S-Poly(T) Plus method.

FIG. 3 shows the difference between the one-step process (the tailing and the reverse transcription were completed in one step) and the two-step process (the tailing was carried out before the reverse transcription) in the Direct S-Poly(T) Plus method.

FIG. 4 shows the initial ratios of the extracted crude RNA added in the Direct S-Poly(T) Plus method.

FIG. 5 shows comparison of the expression levels of miRNAs in serum and plasma of the same volunteer by using Direct S-Poly(T) Plus. ***P<0.001.

FIG. 6 shows comparison of the expression levels of miRNAs in serum and plasma of the same volunteer by using the S-Poly(T) Plus method and using extracted RNAs as templates. miR-cel-54 was used as an internal reference for normalization, ***P<0.001.

FIG. 7 shows the reduction of non-specific amplification by using the hot-start Alpha Taq Polymerase in the Direct S-Poly(T) Plus method.

FIG. 8 shows the amplification plot of hsa-miR-15b-5p. -RT: a negative control without reverse transcriptase. The detection method was Direct S-Poly(T) Plus.

FIG. 9 shows the effect of the amount of the hot-start Alpha taq polymerase on the Ct value of miRNA detection (a 20 μl-system). The detection method was Direct S-Poly(T) Plus.

FIG. 10 show the amplification plot of a miRNA negative control (without reverse transcriptase) (a 20 μl-system) using 0.4 μl of Hotstart Alpha Taq Polymerase. The detection method was Direct S-Poly(T) Plus.

FIG. 11 show the amplification plot of a miRNA negative control (without reverse transcriptase) (a 20 μl-system) using 0.0125 μl of Hotstart Alpha Taq Polymerase. The detection method was Direct S-Poly(T) Plus.

FIG. 12 shows the sensitivity and linear range of the Direct S-Poly(T) Plus method.

FIG. 13 shows comparison of the sensitivity of three miRNA detection methods.

FIG. 14 shows the single-sample validation of six miRNAs that are significantly altered in primary screening of colorectal cancer. The verification method was Direct S-Poly(T) Plus. The data was expressed as ±SE, **P<0.01, ***P<0.001, ns, no significance.

FIG. 15 shows the single-sample validation of six miRNAs that are significantly altered in primary screening of colorectal cancer. The verification method was S-Poly(T) Plus (miR-cel-54 was used as an internal reference). The data was expressed as ±SE, **P<0.01, ***P<0.001, ns, no significance.

### DETAILED DESCRIPTION

In order to better illustrate the problems solved, the technical solutions adopted, and the effects achieved by the present disclosure, the present disclosure will be further described in conjunction with specific embodiments and related materials. It should be noted that the present disclosure includes but is not limited to the following embodiments and combinations thereof.

Unless otherwise stated, the various starting materials used in the following examples were commercially available, and the methods used therein were conventional methods, wherein the primers and probes were available from Integrated DNA Technologies (IDT) Inc., USA.

Sources of major materials used in the present application are as follows:

Blood was obtained from Shenzhen People's Hospital and Peking University Shenzhen Hospital. Plasma was collected as follows: The blood was collected in a blood collection tube containing anticoagulant EDTA, centrifuged at 3,000 rpm, 4° C. for 10 minutes to obtain the supernatant as plasma. The whole blood sample was allowed to stand at room temperature for 1 hour, centrifuged at 3,000 rpm, 4° C. for 10 minutes to obtain the supernatant as serum. The serum/plasma samples were dispensed into 20-50 μl systems and stored at −80° C.

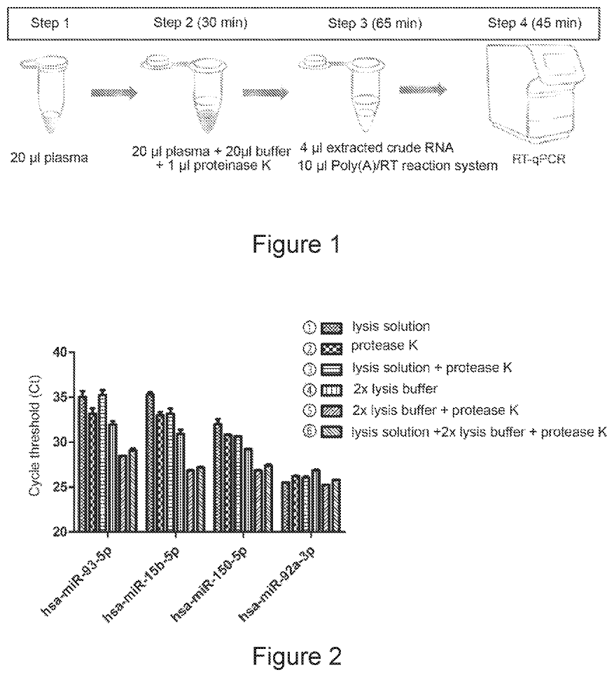

An optimal scheme of the miRNA direct RT-qPCR fluorescence quantitative detection method (Direct S-Poly (T) Plus) is shown in FIG. 1. In the optimal scheme, 35 μl of extracted crude RNA, corresponding to 87.5 μl of cDNA, can be prepared from 20 μl of plasma. To 20 μl qPCR system, 0.5 μl cDNA was added, and 175 miRNAs can be detected on average. The entire miRNA detection process takes only 140 minutes without regard to the operation time.

### Example 1 Comparison of the Contents of Circulating miRNAs in Plasma and Serum by Using the Direct S-Poly(T) Plus Method

In this example, serum and plasma derived from the same volunteer were used as templates simultaneously. 10 pairs of serum and plasma samples were collected from the same healthy volunteer. The expression levels of miRNAs in equivalent amounts of serum or plasma samples were separately measured by using the Direct S-Poly(T) Plus method of the present disclosure. Specifically, the following steps were included.

S1, lysis and centrifugation. The specific steps were as follows:

1) 20 μl of plasma/serum was mixed well with 20 μl of 2×lysis buffer, and 1 μl of proteinase K was added. The mixture was incubated at 50° C. for 20 minutes, then incubated at 95° C. for 5 minutes, and then placed on ice;

2) The mixture was centrifuged at 13,000 g, 4° C. for 5 minutes. The supernatant (extracted crude RNA) was pipetted and transferred to another new centrifuge tube or directly used in S2;

S2, tailing and reverse transcription: the addition of a Poly(A) tail to miRNA and the reverse transcription (synthesis of first-strand cDNA) were carried out in the same reaction system, and S-Poly(T) primer was used in the reverse transcription of miRNA.

The 2×lysis buffer comprised the following components at their final concentrations: 100 mmol/1 Tris-HCl, 300 mmol/1 NaCl, 20 mmol/1 MgCl₂; pH 8.0; and the final concentration of the proteinase K was 15 U/mL.

The reaction system for the tailing and reverse transcription comprised: 4 μL of the extracted crude RNA, 1 μL of 0.05 μM RT primer (reverse transcription primer), 1 U of PolyA polymerase (polyadenylation polymerase), 100 U of MMLV (murine leukemia reverse transcriptase), 1.5 μL of reaction buffer, and supplement of RNase-free water to 10 μL. The reaction buffer comprised the following components at their final concentrations: 200 mM Tris-HCl, 600 mM NaCl, 40 mM MgCl₂, 4 mM ATP, 2 mM dNTP, pH 8.0. The tailing and reverse transcription was carried out as follows. The reaction system was incubated at 37° C. for 30 min, incubated at 42° C. for 30 min, and incubated at 75° C. for 5 min to inactivate enzymes, and then rapidly placed on ice and allowed to stand for 2 min to terminate the inactivation.

The S-Poly(T) primer consisted of four parts. The sequence thereof was, in order from 5′ end to 3′ end, a PCR universal primer sequence of 14 to 20 bases in length, a universal probe sequence of 14 to 20 bases in length, 11 oligo(dT)s, and 5 to 7 specific bases complementary to the 3′ end of miRNA. More preferably, the S-Poly(T) primer sequence was, in order from 5′ end to 3′ end, a PCR universal primer sequence of 16 bases in length, a universal probe sequence of 17 bases in length, 11 oligo (dT)s, and 6 specific bases complementary to the 3′ end of miRNA.

The miRNA sequences detected in the present disclosure were obtained from miRBase. Different S-Poly(T) primers and upstream primers were designed according to respective sequences. S-Poly(T) primer sequences used for detecting different miRNAs are shown in Table 1.

TABLE 1
Primers and probes used in the present disclosure

| miRNA | Forward primer | RT primer |
|---|---|---|
| >hsa-miR-93-5p | GGCAAAGTGCTGTTCGTGC | GTGCAGGGTCCGAGGTCAGAGCCACCTG |
| MIMAT0000093 | | GGCAATTTTTTTTTTTCTAC CT |
| >hsa-miR-15b-5p | CGGTAGCAGCACATCATGG | GTGCAGGGTCCGAGGTCAGAGCCACCTG |
| MIMAT0000417 | | GGCAATTTTTTTTTTTGTAAAC |
| >hsa-miR-150-5p | CCGGGTCTCCCAACCCTTGT | GTGCAGGGTCCGAGGTCAGAGCCACCTG |
| MIMAT0000451 | A | GGCAATTTTTTTTTTTCACTGG |
| >hsa-miR-92a-3p | TTCGGTATTGCACTTGTCCC | GTGCAGGGTCCGAGGTCAGAGCCACCTG |
| MIMAT0000092 | | GGCAATTTTTTTTTTTACAGGC |
| >hsa-miR-451a | CCGGGAAACCGTTACCATTA | GTGCAGGGTCCGAGGTCAGAGCCACCTG |
| MIMAT0001631 | C | GGCAATTTTTTTTTTTAACTCA |
| >hsa-miR-21-5p | TTCGGTAGCTTATCAGACTG | GTGCAGGGTCCGAGGTCAGAGCCACCTG |
| MIMAT0000076 | A | GGCAATTTTTTTTTTTCAACAT |
| >hsa-miR-22-3p | TCGGATCACATTGCCAGGG | GTGCAGGGTCCGAGGTCAGAGCCACCTG |
| MIMAT0000077 | | GGCAATTTTTTTTTTTGGAAAT |
| >hsa-miR-16-5p | TTCGGTAGCAGCACGTAAAT | GTGCAGGGTCCGAGGTCAGAGCCACCTG |
| MIMAT0000069 | A | GGCAATTTTTTTTTTTCGCCAA |
| >hsa-miR-27b-3p | TTCGGTTCACAGTGGCTAAG | GTGCAGGGTCCGAGGTCAGAGCCACCTG |
| MIMAT0000419 | | GGCAATTTTTTTTTTTGCAGAA |
| >hsa-miR-126-3p | GCGGGCGTACCGTGAGTAAT | GTGCAGGGTCCGAGGTCAGAGCCACCTG |
| MIMAT0000445 | | GGCAATTTTTTTTTTTCGCATT |
| >hsa-miR-210-3p | CCGGGCTGTGCGTGTGACA | GTGCAGGGTCCGAGGTCAGAGCCACCTG |
| MIMAT0000267 | GC | GGCAATTTTTTTTTTTCAGCCG |
| >hsa-miR-103a-3p | GGAGCAGCATTGTACAGGG | GTGCAGGGTCCGAGGTCAGAGCCACCTG |
| MIMAT0000101 | | GGCAATTTTTTTTTTTCATAGC |
| >hsa-miR-140-5p | TCGGCAGTGGTTTTACCCTA | GTGCAGGGTCCGAGGTCAGAGCCACCTG |
| MIMAT0000431 | | GGCAATTTTTTTTTTTCTACCA |
| >hsa-miR-124-3p | TCGGTAAGGCACGCGGTG | GTGCAGGGTCCGAGGTCAGAGCCACCTG |
| MIMAT0000422 | | GGCAATTTTTTTTTTTGGCATT |
| >hsa-miR-25-3p | CGGCATTGCACTTGTCTCG | GTGCAGGGTCCGAGGTCAGAGCCACCTG |
| MIMAT0000081 | | GGCAATTTTTTTTTTTCAGACC |
| >hsa-miR-106b-5p | GTCGGTAAAGTGCTGACAGT | GTGCAGGGTCCGAGGTCAGAGCCACCTG |
| MIMAT0000680 | | GGCAATTTTTTTTTTTATCTGC |
| >hsa-miR-423-5p | GTGAGGGGCAGAGAGCGA | GTGCAGGGTCCGAGGTCAGAGCCACCTG |
| MIMAT0004748 | | GGCAATTTTTTTTTTTAAAGTC |
| >hsa-miR-144-3p | TGGTCGGTACAGTATAGATG | GTGCAGGGTCCGAGGTCAGAGCCACCTG |
| MIMAT0000436 | A | GGCAATTTTTTTTTTTAGTACA |
| >hsa-miR-148a-3p | TCGGTCAGTGCATCACAGAA | GTGCAGGGTCCGAGGTCAGAGCCACCTG |
| MIMAT0000243 | | GGCAATTTTTTTTTTTACAAAG |
| >hsa-miR-30b-5p | TTCGGTGTAAACATCCTACA | GTGCAGGGTCCGAGGTCAGAGCCACCTG |
| MIMAT0000420 | C | GGCAATTTTTTTTTTTAGCTGA |
| >cel-miR-54-5p | TCGGAGGATATGAGACGACG | GTGCAGGGTCCGAGGTCAGAGCCACCTG |
| MIMAT0020773 | | GGCAATTTTTTTTTTTGTTCTC |
| Universal reverse | CAGTGCAGGGTCCGAGGT | |
| primer | | |

| | |
|---|---|
| Universal Taqman probe | 56-FAM/CAGAGCCAC/ZEN/CTGGGCAATTT/3IABkFQ |

S3, PCR: a real-time PCR quantitative detection was performed with a specific upstream primer and a universal downstream primer of the miRNA by using the first-strand cDNA obtained from the reverse transcription in step S2 as a template. The specific upstream primer of the miRNA was a miRNA-specific sequence without 3 to 8 bases at the 3′ end, and the universal downstream primer of the miRNA was derived from the universal primer sequence of 14 to 20 bases in length in the S-Poly(T) primer.

The real-time PCR quantitative detection may be performed by using a probe method or a SYBR fluorescent dye method. In the present example, a probe method was used, and the probe used was a universal probe whose sequence was derived from the PCR universal primer sequence of 14 to 20 bases in length in the S-Poly(T) primer. The reaction system for the real-time PCR was as follows:

| Components | Contents |
|---|---|
| 4 × qPCR reaction Buffer (Geneup, μL) | 5 |
| 1 μM Forward Primer (μL) | 4 |
| 10 μM universal reverse primer (μL) | 0.4 |
| 10 μM universal Taqman probe (μL) | 0.5 |
| 100 × ROX Rerference Dye (μL) | 0.2 |
| hotstart Alpha Taq Polymerase (Geneup, μL) | 0.0125 |
| cDNA (μL) | 0.5 |
| RNase-free Water up to (μL) | 20 |

The PCR reaction was performed on an ABI StepOnePlus thermal cycler and the conditions were as follows: 40 cycles of pre-denaturation at 95° C. for 5 minutes, denaturation at 95° C. for 10 s, and annealing at 60° C. for 40 s. Each PCR reaction was carried out in duplicate. Data was analyzed by using GraphPad Prism 5 software and was tested by two-tailed Student's test. The final results were expressed as mean±SD (standard deviation).

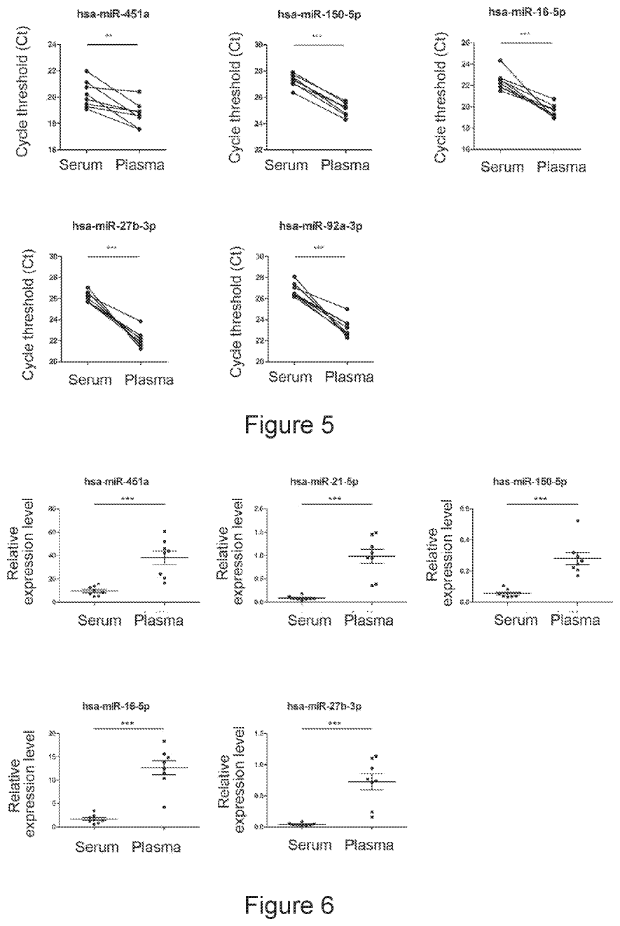

The results showed that both serum and plasma samples could be used in the miRNA direct quantitative RT-qPCR detection, although the detected Ct values of miRNAs in plasma were significantly lower than those in serum, which indicated that the expression levels of miRNAs in plasma were significantly higher than those in serum (FIG. 5).

### Comparative Example 1. Detection of Circulating miRNAs by Using the S-Poly(T) Plus Method

It was needed to extract nucleic acids for detecting circulating miRNAs with the S-Poly(T) Plus method, including the following steps:

(I) Extraction of Total RNA from Serum/Plasma

In this example, total RNA was extracted from serum/plasma with specific steps as follows:

1) To 1 mL of RNAiso-Plus (TaKaRa), 0.1 pM nematode miRNA cel-miR-54 was added in advance as an internal reference and then 100 μL of serum/plasma was added. The mixture was mixed well by pipetting, and allowed to stand at room temperature for 5 minutes. 200 μL of chloroform was added. The centrifuge tube was capped tightly, vortex-mixed for 20 seconds, and then allowed to stand at room temperature for 5 minutes;

2) The resultant mixture was centrifuged at 12,000 g, 4° C. for 15 minutes and then the centrifuge tube was carefully taken out. At this time, the homogenate was divided into three layers, i.e., a colorless supernatant (containing miRNA), an intermediate white protein layer, and a colored lower organic phase. 500 μL of the supernatant was pipetted and transferred into another new 1.5 mL centrifuge tube;

3) To the supernatant, 5 μL of an appropriate concentration of glycogen (Applichem) solution was added at a final concentration of 15 μg/mL, and then an equal volume of isopropanol (505 μL) was added. The mixture was mixed well by inverting it upside-down and allowed to stand at −20° C. or −80° C. for at least 10 minutes;

4) The resultant mixture was centrifuged at 13,500 g, 4° C. for 10 minutes. The supernatant was discarded. The precipitate was washed with 1 mL of 75% ethanol by gently inverting the tube. The tube was centrifuged at 13,500 g, 4° C. for 5 minutes. The supernatant was completely discarded. In a case where a residual solution was stained on the tube wall, a further centrifugation was needed to discard the supernatant completely.

5) The precipitate was dried at room temperature for 2 to 3 minutes and dissolved in 100 μL of RNase-free water. The dissolved product was stored at −80° C. or directly used in miRNA fluorescence quantitative PCR detection.

(II) Detection of miRNAs with the S-Poly (T) Plus Method

miRNAs were detected by using the S-Poly(T) Plus method with the same primers for reverse transcription and for qPCR as those in Example 1 Table 1. The following steps were included:

S1, tailing and reverse transcription: the addition of a Poly(A) tail to miRNA and the reverse transcription (synthesis of first-strand cDNA) were carried out in the same reaction system, wherein S-Poly(T) primers were used for the reverse transcription of miRNA.

The reaction system for the tailing and reverse transcription comprised: 4 μL of total RNA extracted from serum, 1 μL of 0.05 μM RT primer (primer for reverse transcription), 1 U of PolyA polymerase (polyadenylation polymerase), 100 U of MMLV (murine leukemia reverse transcriptase), 2.5 μL of reaction buffer, and supplement of RNase-free water to 10 μL. The reaction buffer comprised 200 mM Tris-HCl, 600 mM NaCl, 40 mM MgCl₂, 4 mM ATP, 2 mM dNTP, pH 8.0. The tailing and reverse transcription were carried out as follows. The reaction system was incubated at 37° C. for 30 min, incubated at 42° C. for 30 min, and incubated at 75° C. for 5 min to inactivate enzymes, and then rapidly placed on ice and allowed to stand for 2 min to terminate the inactivation.

S2. Real-time PCR quantitative detection was performed by using the first-strand cDNA obtained from the reverse transcription in step S1 as a template. A probe method was used in the real-time PCR quantitative detection, and the probe used was a universal probe whose sequence was the same as that in Example 1. The reaction system for the real-time PCR was as follows.

| Components | Contents |
|---|---|
| 4 × qPCR Reaction Buffer (Geneup, μL) | 5 |
| 1 μM Forward Primer (μL) | 4 |
| 10 μM universal reverse primer (μL) | 0.4 |
| 10 μM universal Taqman probe (μL) | 0.5 |
| 100 × ROX Rerference Dye (μL) | 0.2 |
| Hotstart SM Taq Polymerase (Geneup, U) | 0.5 |
| Diluted cDNA (μL) | 0.5 |
| RNase-free Water up to (μL) | 20 |

The PCR reaction was performed on an ABI StepOnePlus thermal cycler and the conditions were as follows: 40 cycles of pre-denaturation at 95° C. for 3 minutes, denaturation at 95° C. for 10 s, and annealing at 60° C. for 30 s. Each PCR reaction was carried out in duplicate. The relative expression level was calculated using 2-\hat{ }ΔCt in this example. Data was analyzed by using GraphPad Prism 5 software and was tested by two-tailed Student's test. The final results were expressed as mean±SD (standard deviation).

The results showed that both serum and plasma samples could be used as a template in detecting miRNA with the S-Poly(T) Plus method. However, the relative expression levels of miRNAs in plasma were significantly higher than those in serum, further confirming that the expression levels of miRNAs in plasma were significantly higher than those in serum (FIG. 6).

### Example 2 Comparison of Different Lysis Schemes Used in the Direct S-Poly(T) Plus (DSPP) Method of the Present Disclosure

In the Direct S-Poly(T) Plus method, miRNAs can be lysed from the protein complex by using any of the following six systems:

(1) lysis system: 20 μl of lysis solution, 20 μl of sample; lysis condition: 75° C. for 5 minutes;

(2) lysis system: 20 μl of RNase-free water, 1 μl of protease K, 20 μl of sample; lysis condition: 50° C. for 20 minutes, then 95° C. for 5 minutes

(3) lysis system: 20 μl of lysis solution, 1 μl of protease K, 20 μl of sample; lysis condition: 50° C. for 20 minutes, then 95° C. for 5 minutes

(4) lysis system: 20 μl of 2×lysis buffer, 20 μl of sample; lysis condition: 75° C. for 5 minutes;

(5) lysis system: 20 μl of 2×lysis buffer, 1 μl of protease K, 20 μl of sample; lysis condition: 50° C. for 20 minutes, then 95° C. for 5 minutes

(6) lysis system: 10 μl of 2×lysis buffer, 10 μl of lysis solution, 1 μl of protease K, 20 μl of sample; lysis condition: 50° C. for 20 minutes, then 95° C. for 5 minutes

The lysis solution used in the above systems comprised the following components at their final concentrations: 2.5% tween-20, 50 mM Tris, and 1 mM EDTA. The 2×lysis buffer comprised the following components at their final concentrations: 100 mmol/1 Tris-HCl, 300 mmol/l NaCl, 20 mmol/l MgCl₂; pH 8.0. The proteinase K had a final concentration of 15 U/mL.

Other operations were the same as in Example 1.

The experimental results showed that in the above schemes, effects when using tween 20 (the main functional component of the lysis solution, see the reference Zhang Q, Oncotarget, 2016, 7(16): 21865-21874) or proteinase K either alone or in combination (corresponding to schemes (1), (2), (3) respectively) were barely satisfactory. In scheme 5 in which a combination of 2×lysis buffer and proteinase K was used, the Ct value of miRNA was the lowest, and was reduced by 0.8˜6.8 as compared to that in scheme 3. When comparing schemes 5 and 6, it can be seen that tween 20 may adversely affect the poly(A)/RT reaction when lysing the miRNA-encapsulating protein complex (FIG. 2). Therefore, the scheme 5 was recommended as the optimal scheme in the present disclosure, and the amount of plasma used in the lysis reaction was 20 to 50 μl.

### Example 3 Comparison of the Sensitivity of an One-Step Process and a Two-Step Process in the Direct S-Poly(T) Plus Method

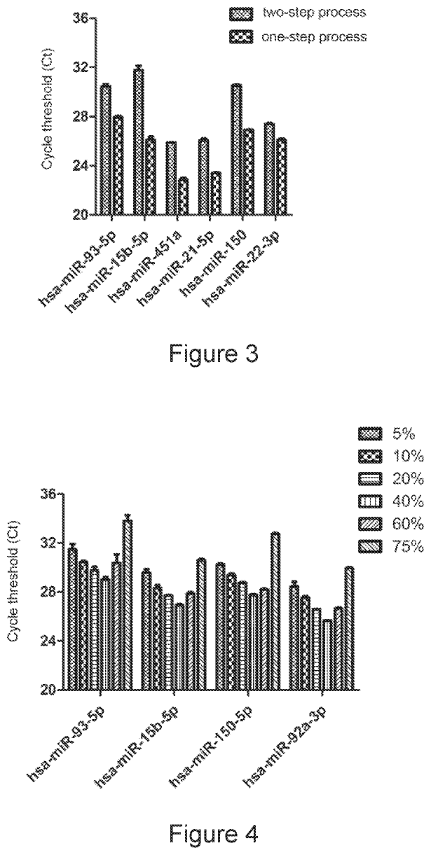

In the S-Poly(T) Plus method described in a previous application (Patent Application Number: 201510558101.5) in which a purified RNA was used as a template, the sensitivity of an one-step process was greatly improved compared to a two-step process. For the two-step process, the Poly (A) tailing of miRNA is carried out before the reverse transcription; and for the one-step process, the Poly (A) tailing and the reverse transcription of miRNA are carried out in the same reaction. In the present disclosure, the sensitivity of the two-step process and the one-step process was compared by using extracted crude RNA as a template and using the same procedure as in Example 1. As shown in FIG. 3, in the Direct S-Poly(T) Plus method, the scheme of the present disclosure increased the sensitivity of the one-step process by 2.5˜52 times (with a difference of 1.7˜5.7 Ct values) as compared with the two-step process thereof (FIG. 3).

### Example 4 Comparison of Effects of Different Proportions of the Extracted Crude RNA Initially Added to the Direct Fluorescence Quantitative PCR Amplification System of miRNA

The extracted crude RNA may contain some components that inhibit the activity of enzymes used in Poly(A) tailing and reverse transcription. So the initial amount of the extracted crude RNA added in the Direct S-Poly(T) Plus method may have an effect on the sensitivity of the method. The experiment was carried out with the same procedure as in Example 1 using different initial amounts of extracted crude RNAs. The results were shown in FIG. 4. It can be seen that Ct values of miRNAs linearly decreased when the initial volume percent of the extracted crude RNA increased from 0.5% to 40%. However, the Ct values of the miRNAs increased when the proportion of the extracted crude RNA as added increased to 60% and 75%. In the present disclosure, an initial amount of 40% of extracted crude RNA was recommended as the optimal ratio.

### Example 5. Test on the Effect of Hot-Start DNA Polymerase in the Direct S-Poly(T) Plus Method

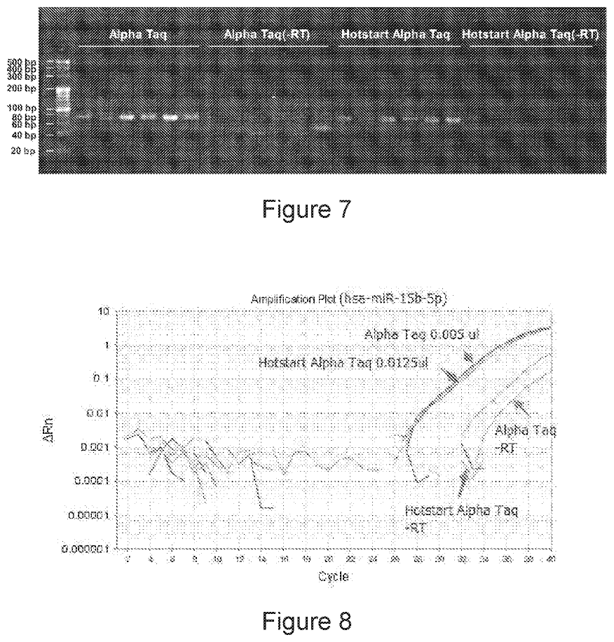

In the Direct S-Poly(T) Plus method system, some genomic DNA contamination may be introduced due to the absence of RNA purification. Therefore, mismatch with genomic DNA is more likely to occur in qPCR. One effective way to reduce non-specific amplification is hot start which prevents or reduces DNA synthesis before the onset of thermal cycling. In this example, the common DNA polymerase and hot-start DNA polymerase used in the PCR procedure in the Direct S-Poly(T) Plus method were compared and analyzed. An effective method to form hot start, i.e., a Taq enzyme antibody, was used in this example. The antibody binds to the DNA polymerase and thus the enzyme cannot be activated before the onset of thermal cycling. The hot-start DNA polymerase used in this example was Hotstart Alpha Taq Polymerase. The specific preparation was as follows. Alpha Taq Polymerase (VitaNavi, St. Louis USA) and Taq Antibody (Fei Peng, Shenzhen) were mixed in equal volumes and the mixture was left at room temperature for 6 hours. As can be seen from FIGS. 7 and 8, the use of a hot start enzyme effectively reduced non-specific amplification.

### Example 6. Effect of the Amount of Hotstart Alpha Taq Polymerase on the Amplification Efficiency of the Direct S-Poly(T) Plus Method

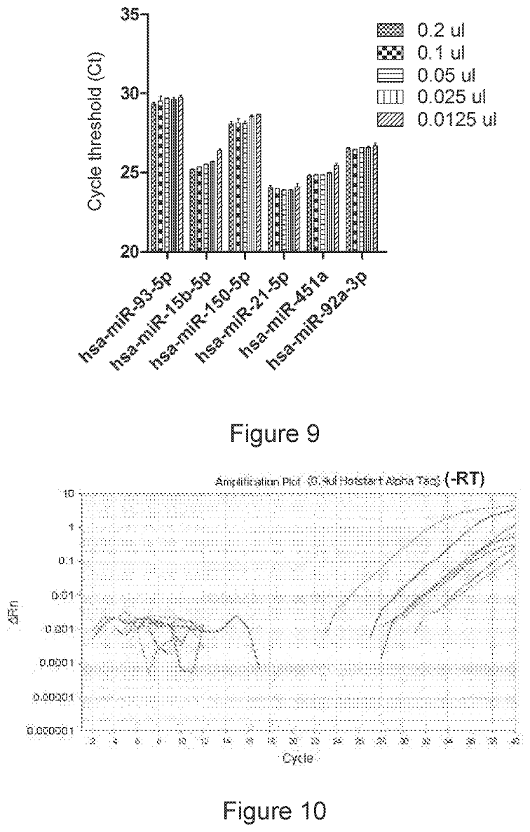

In this example, the effect of the amount of Hotstart Alpha Taq Polymerase on the direct amplification efficiency of miRNA was analyzed. As can be seen from FIG. 9, the Hotstart Alpha Taq Polymerase had a very high activity, and an amount of 0.0125 μl of enzyme can meet the requirements for the amplification of a 20 μl-PCR system.

### Example 7. Effect of the Amount of Hotstart Alpha Taq Polymerase on the Non-Specific Amplification Occurred in the Direct S-Poly(T) Plus Method

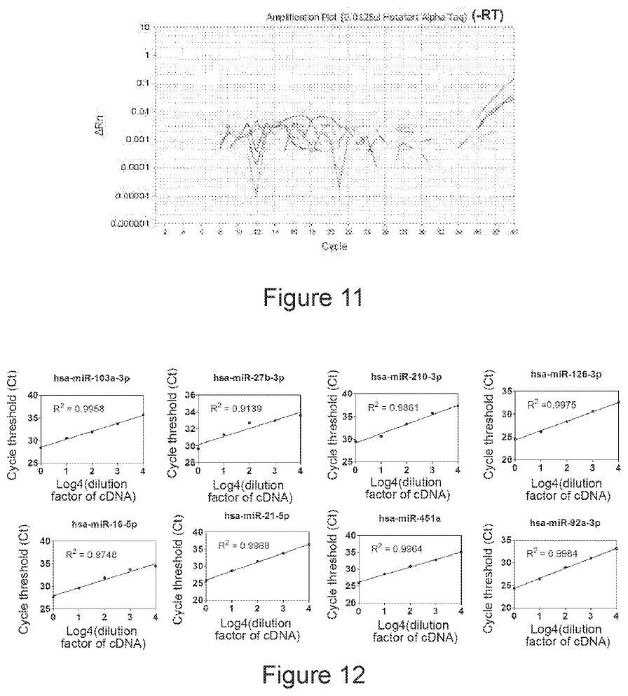

The effect of different amounts of Hotstart Alpha Taq Polymerase on non-specific amplification was explored in this example. As can be seen from FIG. 10, adding 0.4 μl of Hotstart Alpha Taq Polymerase to a 20 μl-PCR system caused non-specific amplification, to some extent. When the amount of enzyme was reduced to 0.0125 μl (as shown in FIG. 11), the non-specific amplification was well suppressed.

### Example 8. The Linear Gradient Range in Detecting Plasma miRNAs by the Direct S-Poly(T) Plus Method

The linear gradient range in detecting plasma miRNAs by the Direct S-Poly(T) Plus method was analyzed in the example. The serum RNA was subjected to 4-fold serial dilution (the amount of the initial plasma corresponding to the amount of the total RNA was 0.1-0.0004 μl), and then detected. As seen from FIG. 12, a good linear correlation coefficient R2 (0.9139-0.9988) was obtained in detecting plasma miRNAs (hsa-miR-451a, hsa-miR-21-5p, hsa-miR-126-3p, hsa-miR-92a-3p, hsa Both -miR-210-3p, hsa-miR-27b-3p, hsa-miR-103a-3p and hsa-miR-92a-3p) by the Direct S-Poly(T) Plus method. Therefore, the detection of plasma miRNAs by the Direct S-Poly(T) Plus method has a good linear relationship and a wide dynamic range.

### Example 9. Comparison of the Direct S-Poly (T) Plus Method and Other Methods

In this example, the Direct S-Poly(T) Plus method was compared with the most popular Stem-loop method and the S-Poly(T) Plus method in Comparative Example 1. A purified RNA was used as a template in the Stem-loop method and the S-Poly(T) Plus method. The S-Poly(T) Plus method was performed the same as in Example 1, and the Stem-loop method was performed according to the instructions of the TaqMan microRNA assay kit (Applied Biosystems).

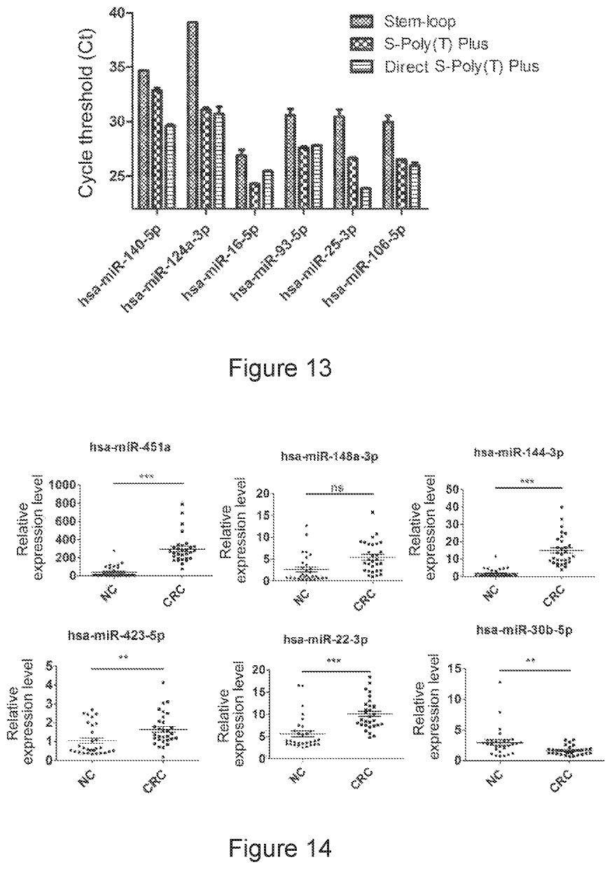

In this example, six miRNAs, i.e., hsa-miR-140-5p, hsa-miR-124a-3p, hsa-miR-16-5p, hsa-miR-93-5p, hsa-miR-25-3p, and hsa-miR-106-5p, were detected by the three miRNA detection methods. As shown in FIG. 13, except that the Ct values of hsa-miR-16-5p (25.43) and hsa-miR-93-5p (27.78) detected by S-Poly(T) Plus method were slightly lower, the Ct values of other miRNAs detected by Direct S-Poly(T) Plus method were the lowest. The Direct S-Poly(T) Plus method was 7-342 times more sensitive (2.8-8.4 Ct values) than the stem-loop method.

### Example 10. Analysis of miRNA Expression Profiles in Colorectal Cancer Patients by the Direct S-Poly(T) Plus Method

In this example, single-sample verification was performed for six miRNAs by using the Direct S-Poly(T) Plus method. The internal reference hsa-miR-93-5p was used for normalization. Plasma samples used were derived from 30 healthy volunteers and 30 colorectal cancer patients. As can be seen from FIG. 14, the expression levels of hsa-miR-22-3p, hsa-miR-423-5p, hsa-miR-144-3p, and hsa-miR-451a were significantly up-regulated in the single-sample verification, the expression level of hsa-miR-30b-5p was significantly down-regulated, and the expression level of hsa-miR148a-3p was not significantly changed.

### Comparative Example 2. Analysis of miRNA Expression Profiles in Colorectal Cancer Patients by the S-Poly(T) Plus Method

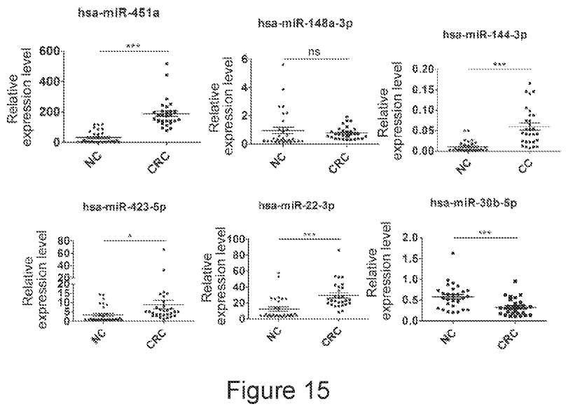

In this example, single-sample verification was again performed for six miRNAs by using the S-Poly(T) Plus method in order to verify the conclusions obtained by the Direct S-Poly(T) Plus method. The external reference cel-miR-54 was used for normalization. The used plasma samples were the same as in Example 10. As can be seen from FIG. 15, the expression trends of the six miRNAs were consistent with those obtained by the Direct S-Poly(T) Plus method. Therefore, we can conclude that the Direct S-Poly(T) Plus method is stable and reliable, and hsa-miR-22-3p, hsa-miR-423-5p, hsa-miR-144-3p, hsa-miR-451a, and hsa-miR-30b-5p can be used as potential biomarkers for colorectal cancer.

The present disclosure introduces a sensitive miRNA detection method, that is, the direct fluorescent quantitative PCR amplification technology of miRNA (Direct S-Poly(T) Plus, abbreviated as DSPP), which is based on the S-Poly(T) Plus technology and does not require RNA extraction. In the Direct S-Poly(T) Plus method, the miRNA is firstly released from the protein complex to obtain an extracted crude RNA; then, based on the S-Poly(T) Plus method, the extracted crude RNA is simultaneously subjected to tailing and reverse transcription in the same reaction system. cDNA can be prepared within 95 minutes by using the Direct S-Poly(T) Plus method of the present disclosure without regard to the operation time, and the entire miRNA detection process can be completed within 140 minutes, including the period for qPCR. It takes only 3 hours to complete the entire operation process for detecting one miRNA from 48 samples, while the method involving nucleic acid extraction takes at least one day. This Direct S-Poly(T) Plus technology will greatly simplify the detection process, reduce costs, and more effectively promote the early entry of circulating miRNA as tumor markers into clinical applications.

The above-mentioned embodiments, the description of which is relatively specific and detailed, are merely illustrative of several embodiments of the present disclosure and should not be construed as limiting the scope of the present disclosure. It should be noted that a number of variations and modifications may be made by those skilled in the art without departing from the spirit and scope of the present disclosure and these variations and modifications are fall within the scope of the present disclosure. Therefore, the scope of the present disclosure should be determined by the appended claims.

1. An RT-qPCR-based method for directly and quantitatively detecting a circulating miRNA, comprising the following steps:
S1, lysis and centrifugation: fully lysing a protein complex in a sample with a lysis reagent to release the miRNA from the sample; centrifuging briefly to obtain a supernatant as extracted crude RNA;
S2, tailing and reverse transcription: adding a Poly(A) tail to the extracted crude RNA obtained in step S1 and performing S-Poly (T) specific reverse transcription;
S3, RT-qPCR quantitative detection: performing RT-qPCR quantitative detection by using the reverse transcription product cDNA obtained in step S2 as a template.

2. The detection method according to claim 1, wherein the lysis reagent in step S1 comprises components: 20 μl of 2×lysis buffer, 1 μl of protease K, and is used to process 20 μl of the sample.

3. The detection method according to claim 2, wherein the 2×lysis buffer comprises the following components at their final concentrations: 100 mM Tris-HCl, 300 mM NaCl, 20 mM MgCl₂; pH 8.0.

4. The detection method according to claim 2, wherein the final concentration of the proteinase K is 15 U/mL.

5. The detection method according to claim 1, wherein the reaction conditions for the lysis reagent in step S1 is 50° C. for 20 minutes and then 95° C. for 5 minutes.

6. The detection method according to claim 1, wherein the centrifugation in step S1 is carried out at 10,000 to 14,000 g, 4° C. for 5 to 15 minutes.

7. The detection method according to claim 1, wherein the volume percentage of the extracted crude RNA as a template added to the reaction system for the tailing and reverse transcription in step S2 is 5 to 75%.

8. The detection method according to claim 1, wherein the reaction system for tailing and reverse transcription in step S2 comprises: 0.5-7.5 μL of a supernatant template, 1±0.2 μL of 0.5 μmol/L RT primer, 1±0.2U of PolyA Polymerase, 100±20U of MMLV, and 2.375-0.625 μL of reaction buffer, and RNase-free Water is added to 10 μL; the tailing and reverse transcription is carried out under conditions as follows: incubating the reaction system at 37˜42° C. for 50˜70 min, at 74˜76° C. for 3-7 minutes to inactivate enzymes, then quickly placing the reaction system on ice and allowing the same to stand for 2 min to stop the inactivation.

9. The detection method according to claim 1, wherein the reaction system for the real-time PCR in step S3 is: 5 μL of 4×qPCR reaction Buffer, 4 μL of 1 μmol/L Forward Primer, 0.4 μL of 10 μmol/L universal reverse Primer, 0.5 μL of 10 μmol/L universal Taqman probe, 0.2 μL of 100×ROX Reference Dye, 0.0125 μL of Hotstart Alpha Taq Polymerase, 0.5 μL of cDNA, and supplement of RNase-free Water to 20 μL; the reaction is performed under conditions as follows: 40 cycles of pre-denaturation at 95° C. for 5 minutes, denaturation at 95° C. for 10 s, and annealing at 60° C. for 40 s.

10. The detection method according to claim 1, wherein the sample is plasma, serum, urine, tears, milk, saliva, sputum, or stool extraction supernatant.
